(19) United States
(12) Patent Application Publication
(10) Pub. No.: US 2026/0365728 A1
(43) Pub. Date: Nov. 19, 2026

(54) Bispecific Antibody Targeting CD3 and CD20 and Use Thereof

(71) Applicants: Yang Yang, Yangpu (CN); Wei Yin, Yangpu (CN)

(72) Inventors: Yang Yang, Yangpu (CN); Wei Yin, Yangpu (CN)

(21) Appl. No.: 19/689,071

(22) Filed: May 27, 2026

Related U.S. Application Data
(63) Continuation of application No. 17/636,440, filed on Jul. 23, 2020, now Pat. No. 12,662,536.

(30) Foreign Application Priority Data
Aug. 19, 2019 (CN) 201910764107.6

(51) Int. Cl. C07K 16/28; A61K 39/00; A61P 35/02; C07K 16/46

(52) U.S. Cl. CPC C07K 16/2809; A61P 35/02; C07K 16/2887; C07K 16/468; A61K 2039/505; C07K 2317/31; C07K 2317/55; C07K 2317/73; C07K 2317/92

(57) ABSTRACT

The present invention provides an anti-CD3 single chain Fab molecule and an anti-CD20 single chain Fab molecule and their use in preparation of a bispecific antibody. The present invention also provides a bispecific antibody targeting CD3 and CD20 and its use in preparation of a medicament for treatment of a B-cell related disease.

### CROSS-REFERENCE TO RELATED APPLICATIONS

This application is a continuation of U.S. application Ser. No. 17/636,440, filed Feb. 18, 2022, which is a national stage application under 35 U.S.C. § 371 of International Application No. PCT/CN2020/103731, filed Jul. 23, 2020, which claims priority to Chinese Patent Application No. 201910764107.6, filed Aug. 19, 2019, the entire contents of each of which are incorporated herein by reference.

### SEQUENCE LISTING

The material in the XML file named “P10497-PI-US-CON_260722_Sequence_Listing.xml”, created on Jul. 22, 2026, and having a size of 17,333 bytes, is incorporated by reference herein.

### FIELD OF THE INVENTION

The present invention relates to an anti-CD3 single chain Fab molecule and an anti-CD20 single chain Fab molecule, and also to a bispecific antibody targeting CD3 and CD20 and use thereof.

### BACKGROUND OF THE INVENTION

B-cell lymphoma is a common hematologic disorder classified as Hodgkin's lymphoma and non-Hodgkin's lymphoma. Its etiology is unknown, and immunodeficiency and environmental factors are considered as possible factors in its development. The treatment and prognosis of B-cell lymphoma depends on the specific type of lymphoma and the stage classification. Depending on the clinical progression, B-cell lymphoma is classified as inert lymphomas and aggressive lymphomas. Inert lymphomas usually progress slowly and maintain stable and long-term survival for many years, but are not curable, while aggressive lymphomas usually require immediate and urgent treatment. The current treatment of B-lymphocytoma is not satisfactory due to the lack of effective therapeutic drugs.

CD3 is a differentiation antigen expressed only on the surface of T cells linked to the T cell receptor (TCR) and is a molecule necessary for T cell activation. CD3 includes four chains, ε, ζ, δ and γ, while functional CD3 is a dimer formed by two of these chains. An anti-CD3 antibody can bind to CD3 on the surface of T cells, producing effects similar to those of the TCR-CD3 molecule and thus activating T lymphocytes.

CD20 is a B-cell marker expressed in mature and activated B-lymphocytes and is widely expressed in B-cell non-Hodgkin's lymphoma and other B-cell malignancies, but not in cells such as precursor B-lymphocytes, plasma cells and lymphoid pluripotent stem cells. In addition, CD20 antigen is clearly revealed and easily identified, and no free CD20 is present in serum in the body. Therefore, CD20 is a good therapeutic target for B-lymphocytoma.

Since CD3 and CD20 are specific targets for T and B lymphocytes with good drug-forming potential, several research teams have constructed or are constructing bispecific antibodies targeting CD3 and CD20, and some have applied for patents (e.g. CN104640881A and CN104558191A), but due to the limitations of molecular structure and development technology, bispecific antibodies usually suffer from low expression, heavy chain mismatch, decreased binding of antibody and antigen, and cumbersome purification processes.

### SUMMARY OF THE INVENTION

In an aspect, the present invention provides an anti-CD3 single chain Fab molecule comprising a Fab light chain and a Fab heavy chain, wherein C-terminal of the Fab light chain is connected to N-terminal of the Fab heavy chain by a linking peptide having a length of 45 to 80 amino acids.

In some embodiments, the linking peptide has a length of 60 amino acids.

In some embodiments, the linking peptide comprises a tandemly repeated GGGGS (SEQ ID NO: 11) sequence.

In some embodiments, the anti-CD3 single chain Fab molecule comprises an amino acid sequence having at least 90% identity to the sequence as set forth in SEQ ID NO: 6 or 9.

In another aspect, the present invention provides an anti-CD20 single chain Fab molecule comprising a Fab light chain and a Fab heavy chain, wherein C-terminal of the Fab light chain is connected to N-terminal of the Fab heavy chain by a linking peptide having a length of 45 to 80 amino acids.

In some embodiments, the linking peptide has a length of 60 amino acids.

In some embodiments, the linking peptide comprises a tandemly repeated GGGGS sequence.

In some embodiments, the anti-CD20 single chain Fab molecule comprises an amino acid sequence having at least 90% identity to the sequence as set forth in SEQ ID NO: 10.

In another aspect, the present invention provides use of the anti-CD3 single chain Fab molecule or the anti-CD20 single chain Fab molecule for preparation of a bispecific antibody.

In another aspect, the present invention provides a bispecific antibody comprising a first moiety for binding to CD3 and a second moiety for binding to CD20, wherein the first moiety is an anti-CD3 single chain Fab molecule comprising a Fab light chain and a Fab heavy chain with C-terminal of the Fab light chain being connected to the N-terminal of the Fab heavy chain by a linking peptide having a length of 45 to 80 amino acids; the second moiety comprises a light chain and a heavy chain of an anti-CD20 antibody.

In some embodiments, the linking peptide has a length of 60 amino acids.

In some embodiments, the linking peptide comprises a tandemly repeated GGGGS sequence.

In some embodiments, the first moiety for binding to CD3 comprises an amino acid sequence having at least 90% identity to the sequence as set forth in SEQ ID NO: 6 or 9.

In some embodiments, the light chain of the anti-CD20 antibody comprises an amino acid sequence having at least 90% identity to the sequence as set forth in SEQ ID NO: 7, and the heavy chain of the anti-CD20 antibody comprises an amino acid sequence having at least 90% identity to the sequence as set forth in SEQ ID NO: 8.

In some embodiments, C-terminal of the light chain of the anti-CD20 antibody is linked to N-terminal of the heavy chain of the anti-CD20 antibody by a linking peptide.

In some embodiments, the second moiety for binding to CD20 comprises an amino acid sequence having at least 90% identity to the sequence as set forth in SEQ ID NO: 10.

In another aspect, the present invention provides use of the bispecific antibody in preparation of a medicament for treatment of a B-cell related disease.

In some embodiments, the B-cell related disease is B-cell leukemia or B-cell lymphoma.

In another aspect, the present invention provides a pharmaceutical composition comprising the bispecific antibody and a pharmaceutically acceptable carrier.

In another aspect, the present invention provides a method of treating a B-cell related disease in a patient comprising administering to the patient a therapeutically effective amount of the bispecific antibody or the pharmaceutical composition.

### BRIEF DESCRIPTION OF THE DRAWINGS

FIG. 1 shows a schematic structure of the candidate molecule prepared in Example 1 for assessing the effect of the linking peptide on the properties of the single-chain Fab molecule.

FIG. 2 shows the results of SDS-PAGE electrophoresis analysis of the above candidate molecules with different length linking peptides expressed in cells and purified.

FIG. 3 shows the results of the assay of CD3 binding ability of the above candidate molecules with different lengths of linking peptides.

FIG. 4 shows the structure of the bispecific antibodies YY0421 and YY0422 of the present invention. α-CD3 refers to the CD3-binding moiety and α-CD20 refers to the CD20-binding moiety.

FIG. 5 shows the results of Western blotting assays of the bispecific antibody of the present invention and the bispecific antibody REGN1979 as a control (Con in the Figure) bound to CD3 (FIG. 5a) and CD20 (FIG. 5b).

FIG. 6 shows the results of the assay for binding abilities of the bispecific antibody of the present invention and the bispecific antibody REGN1979 as a control to Raji cells.

FIG. 7 shows the results of the assay for binding ability of the bispecific antibody of the present invention and the bispecific antibody REGN1979 as a control to Jurkat cells.

FIG. 8 shows the results of killing of human Daudi cells (FIG. 8) by the bispecific antibody of the present invention and the bispecific antibody REGN1979 as a control. The vertical coordinate indicates the value of relative change in proliferation.

FIG. 9 shows the results of the inhibition of the growth of human Daudi cell-induced subcutaneous tumors in nude mice by the bispecific antibody of the present invention as well as the bispecific antibody REGN1979 as a control. The horizontal coordinate indicates the number of days and the vertical coordinate indicates the tumor volume (cubic millimeters).

### DETAILED DESCRIPTION OF THE INVENTION

Unless otherwise indicated, all technical and scientific terms used herein have the meanings commonly understood by those of ordinary skill in the art.

“Antibodies” are immunoglobulins secreted by plasma cells (effector B cells) and used by the body's immune system to neutralize foreign substances (peptides, viruses, bacteria, etc.). The foreign substance is accordingly called an antigen. The basic structure of a classical antibody molecule is a tetramer consisting of two identical heavy chains and two identical light chains. The heavy chain is divided into a variable region located at the amino terminus (VH) and a constant region located at the carboxyl terminus (CH) based on the difference in conserved amino acid sequences. The light chain is similarly divided into a variable region (VL) located at the amino terminus and a constant region (CL) located at the carboxyl terminus. The variable region of a heavy chain and a light chain interact to form the antigen binding site (Fv). The constant region of the light chain includes only one Ig domain, while the constant region of the heavy chain includes different numbers of Ig domains depending on the type of antibody (Isotype). For example, IgG, IgA, and IgD include three Ig domains: CH1, CH2, and CH3, while IgM and IgE include four Ig domains: CH1, CH2, CH3, and CH4.

“Fab” refers to the fragment of antigen binding, which consists of an intact light chain of the antibody and the VH and CH1 structural domains of the heavy chain. In some embodiments of the present invention, the C-terminal of the light chain of Fab is linked to the N-terminal of the heavy chain by a short linking peptide to form a “single chain Fab (scFab) molecule”. For the convenience of description, the light chain portion of the single chain Fab molecule is referred to as the “Fab light chain” and the heavy chain portion is referred to as the “Fab heavy chain” in some contexts where Fab is mentioned. In some embodiments, the Fab heavy chain may also include an Fc fragment attached at the C-terminal.

The “linking peptide”, which is described in more detail herein in examples, may have a length of 40 to 80 (e.g., 50, 52, 55, 58, 60, 62, 65, 70, 75, or 78) amino acids. Use of linking peptides helps stabilize the molecular structure and prevent light chain mismatches in bispecific antibodies.

The “Fc fragment” is a fragment crystallizable region, which corresponds to the CH2 and CH3 structural domains of IgG antibody. IgG can bind to cells with corresponding receptors on their surface through its Fc fragment to produce different biological effects, such as opsonization and antibody-dependent cell-mediated cytotoxic effects (ADCC). The antibody Fc fragment can be obtained by hydrolysis of the antibody molecule by protein hydrolases (e.g. papain). In some embodiments, the “Fc fragment” may also comprise a hinge region of the antibody heavy chain. In some embodiments, the “Fc fragment” is derived from IgG, IgA, IgD, or IgM antibodies.

In some embodiments, a single-chain Fab molecule for binding CD3 acts as a CD3-binding moiety to bind to another antigen-binding moiety to form a bispecific antibody targeting CD3 and that other antigen. In some embodiments, the single-chain Fab molecule acts as a CD3-binding moiety to bind to a CD20-binding moiety to form a bispecific antibody targeting CD3 and CD20. In some embodiments, the single-chain Fab molecule acts as a CD3-binding moiety to bind to a variety of additional antigen-binding moieties to form multispecific antibodies.

In some embodiments, a single-chain Fab molecule for binding CD20 acts as a CD20-binding moiety to bind to another antigen-binding moiety to form a bispecific antibody targeting CD20 and that other antigen. In some embodiments, the single-chain Fab molecule acts as a CD20-binding moiety to bind to a CD3-binding moiety to form a bispecific antibody targeting CD3 and CD20. In some embodiments, the single-chain Fab molecule acts as a CD20-binding moiety to bind to a variety of additional antigen-binding moieties to form multispecific antibodies.

The binding of the CD20-binding moiety to the CD3-binding moiety of the bispecific antibody of the present invention may be covalent (e.g., through the formation of disulfide bonds between Fc fragments) or non-covalent.

In some embodiments, the light and heavy chain variable structural domains of the CD3-binding moiety of the bispecific antibody are derived from OKT3 monoclonal antibody or L2K monoclonal antibody (WO2004106380). In some embodiments, the light and heavy chain variable structural domains of the CD20-binding moiety of the bispecific antibody are derived from Rituximab or Ofatumumab.

When it is referred that an antibody “binds” to an antigen, it means that the antibody is capable of recognizing and detectably binding to that antigen. The binding of an antibody to an antigen can be determined by a well-known antigen-antibody binding assay, such as an ELISA. In addition, the dissociation equilibrium constant for antibody-antigen binding, KD, can be used to express the binding affinity of the antibody to the corresponding antigen.

In some embodiments of the present invention, the “Knobs-into-holes” structure of the Fc fragment is used to prevent heavy chain mismatches in bispecific antibodies. This technology was developed by Genentech (see U.S. Pat. No. 5,731,168), which is done by mutating a threonine (T) with a smaller volume at position 366 in the heavy chain CH3 region of one of the antibodies to a tyrosine (Y) with a larger volume, forming a prominent “knob” structure (T366Y, (Kabat numbering system)); while mutating a tyrosine (Y) with a larger volume at position 407 in the heavy chain CH3 region of the other antibody to a threonine (T) with small volume, forming a concave “hole” structure (Y407T). The correct assembly between two different antibody heavy chains is achieved by exploiting the spatial site blocking effect of the “Knobs-into-holes” structure. After the mutation, the product correct assembly rate has improved significantly and can meet the requirements of large-scale production. However, the modification of the heavy chain CH3 reduces the stability of the antibody structure. To overcome the drawback, a more stable “3+1” pattern is constructed by random mutation screening through phage display technique, i.e., the T366W mutation formed a prominent “knob” type and three amino acid mutations (T366S, L368A and Y407V) formed a concave “hole” type. The Knobs-holes structure is designed to facilitate the assembly of two heterologous antibody heavy chains.

“Sequence identity” refers to the degree of similarity between amino acid or nucleotide sequences and is generally expressed as a percentage of identity, which can be determined by visual inspection or by computer programs (e.g. BLAST). In some embodiments of the present invention, the single-stranded Fab molecule for binding to CD3 comprises the amino acid sequence as set forth in SEQ ID NO: 6, or comprises an amino acid sequence having at least 80% (e.g., at least 90%, at least 95%, at least 98%, or at least 99% or 100%) sequence identity to the sequence as set forth in SEQ ID NO: 6.

In some embodiments of the present invention, the CD3-binding moiety of the bispecific antibody comprises the amino acid sequence as set forth in SEQ ID NO: 6 or comprises an amino acid sequence having at least 80% (e.g., at least 90%, at least 95%, at least 98%, at least 99%, or 100%) sequence identity to the sequence as set forth in SEQ ID NO: 6. In some embodiments of the present invention, the light chain of the CD20-binding moiety of the bispecific antibody comprises the amino acid sequence as set forth in SEQ ID NO: 7, or comprises an amino acid sequence having at least 80% (e.g., at least 90%, at least 95%, at least 98%, at least 99%, or 100%) sequence identity with the sequence as set forth in SEQ ID NO: 7; the heavy chain of the CD20-binding moiety of the bispecific antibody comprises the amino acid sequence as set forth in SEQ ID NO: 8, or comprises an amino acid sequence having at least 80% (e.g., at least 90%6, at least 95%, at least 98%, at least 99%, or 100%) sequence identity to the sequence as set forth in SEQ ID NO: 8.

In some embodiments of the present invention, the CD3-binding moiety of the bispecific antibody comprises the amino acid sequence as set forth in SEQ ID NO: 9 or comprises an amino acid sequence having at least 80% (e.g., at least 90%, at least 95%, at least 98%, at least 99%, or 100%) sequence identity to the sequence as set forth in SEQ ID NO: 9. In some embodiments of the present invention, the CD20-binding moiety of the bispecific antibody comprises the amino acid sequence as set forth in SEQ ID NO: 10 or comprises an amino acid sequence having at least 80% (e.g., at least 90%, at least 95%, at least 98%, at least 99%, or 100%) sequence identity to the sequence as set forth in SEQ ID NO: 10.

It will be understood by those skilled in the art that, on the basis of the specific sequences provided herein, the corresponding variants of the single-chain Fab molecules or bispecific antibodies for binding to CD3 or CD20 provided herein can be obtained by substituting, deleting, or adding a plurality of amino acids and verifying or screening the binding capacity or biological activity of the resulting products with the corresponding antigens, which shall also be included in the scope of the present invention.

When referring to pharmaceutical compositions, the term “pharmaceutically acceptable carrier” is used to refer to a solid or liquid diluent, filler, antioxidant, stabilizer, etc., that can be safely administered and is suitable for human and/or animal administration without undue adverse side effects and is suitable for maintaining the viability of the drug or active agent located therein.

The term “therapeutically effective amount” means an amount of an active compound that is sufficient to elicit a biological or medical response in a subject as desired by the clinician. A “therapeutically effective amount” of the bispecific antibody of the present invention can be determined by those skilled in the art based on route of administration, weight, age, and condition, etc. of the subject. For example, a typical daily dose can range from 0.01 mg to 100 mg of active ingredient per kg of body weight.

The present invention is further described below in combination with specific examples.

### Example 1. Screening of Lengths of Flexible Linking Peptides Used to Construct the Anti-CD3 Single Chain Fab Molecules

Based on the spatial structure of the Fab molecule, the inventors designed a single-chain Fab molecule targeting CD3, i.e., the light chain and its corresponding heavy chain variable region, as well as the CH1 fragment and the “knob” Fc fragment, were linked by a flexible linking peptide, in which the light chain and heavy chain variable region sequences were derived from the L2K antibody. The resulting single chain Fab molecule had the amino acid sequence as set forth in SEQ ID NO: 6.

Considering that the length of the flexible linking peptide should be not less than 3.5 nm and the distance between adjacent peptide bonds of amino acids is 0.38 nm, and considering that the spatial structure of scFab needs to maintain a certain flexibility, we designed several flexible linking peptides with lengths between 26 and 80 amino acids, and the lengths and sequences of five representative linking peptides are shown in Table 1.

TABLE 1
Lengthes and sequences of flexibly linking peptides

| | Number of amino | |
|---|---|---|
| SEQ | acids of flexibly | Amino acid |
| ID NO | linking peptides | sequences of flexibly linking peptides |
| 1 | 26 | GGGSGGSGGSGGSGGSGGSGGSGGSG |
| 2 | 32 | GGGGSGGGGSGGGGSGGGGSGGGGSGGGGSGG |
| 3 | 45 | GGGGSGGGGSGGGGSGGGGSGGGGSGGGGSGGGGSGGGGS |
| | | GGGGS |
| 4 | 60 | GGGGSGGGGSGGGGSGGGGSGGGGSGGGGSGGGGSGGGGS |
| | | GGGGSGGGGSGGGGSGGGGS |
| 5 | 80 | GGGGSGGGGSGGGGSGGGGSGGGGSGGGGSGGGGSGGGGS |
| | | GGGGSGGGGSGGGGSGGGGSGGGGSGGGGSGGGGSGGGGS |

### 1.1 Effect of Different Lengths of Flexible Linking Peptides on Expression Levels

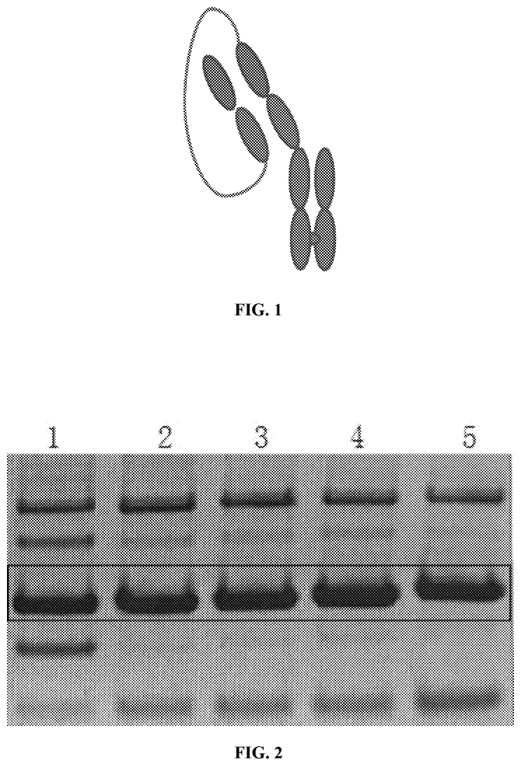

The nucleic acid sequences encoding the above single-stranded Fab molecules and the “hole” Fc fragment were synthesized by overlapping PCR techniques and cloned into the eukaryotic expression vector pcDNA (purchased from Life Technologies, Inc.), respectively. Then, the DNA-transfection reagent complex was prepared by mixing the expression vector including the nucleic acid sequence encoding the single-stranded Fab molecule and the expression vector including the sequence encoding the “hole” Fc fragment at a ratio of 1.5:1 to a mixture of 200 μg in total, and then mixing with the transfection reagent PEI (polyethylenimine) at a mass to volume ratio of 1:2.5. The DNA-transfection reagent complex was added dropwise to 200 mL of 293F cells in logarithmic growth phase. The cell supernatant was collected 5-7 days after transfection, filtered through a 0.45 μm membrane, and added to an affinity chromatography gel column (Protein A) rinsed with binding buffer (12.15 g of Tris (Tris(hydroxymethyl)aminomethane) dissolved in sterilized water, pH adjusted to 7.0, 8.78 g of NaCl added, and volume balanced to 1 L), followed by elution solution (7.5 g glycine dissolved in double-distilled water, pH 3.5, 8.78 g NaCl, volume balanced to 1 L). The column was then eluted with eluent (7.5 g of glycine dissolved in double-distilled water, pH adjusted to 3.5, 8.78 g of NaCl added, and volume balanced to 1 L). The elution fraction was neutralized with 1 M Tris-HCl (Tris(hydroxymethyl)aminomethane-HCl), pH 9.0 to obtain candidate molecules with different lengths of flexible linking peptide linkages (structures are shown schematically in FIG. 1).

After taking 20 μL of eluate and adding the sample loading buffer, the protein was denatured at 95 °C for 10 minutes and separated by polyacrylamide gel electrophoresis, and the bands were shown by Coomassie Brilliant Blue staining. The staining results are shown in FIG. 2. Lanes 1 to 5 correspond to candidate molecules comprising flexible linking peptides of 26, 32, 45, 60 and 80 amino acids in length, respectively. As seen in the figure, the candidate molecule constructed using the flexible linking peptide of 60 amino acids in length (the bands marked by black boxes in the figure, Fab single chain molecule containing the Fc fragment) had slightly higher expression and fewer impurity bands.

### 1.2 Effect of Different Lengths of Flexible Linking Peptides on Thermal Stability

The purified proteins (candidate molecules) from part 1.1 above were subjected to heat treatment at 37 °C for 24 hours and 50 °C for 2 hours, respectively, and were subjected to SEC-HPLC along with the antibodies placed at 4 °C for the same treatment time. The operation of SEC-HPLC detection is briefly described as follows: TSKgel® G3000SWXL gel column (TOSOH Corporation) was selected and the column was equilibrated with the mobile phase (50 mM phosphate buffer, 150 mM NaCl, pH 7.0) followed by UV detection. 50 μL of each protein treated at different temperatures was passed through the column at a flow rate of 1 mL/min, and each sample was run for 20 minutes to fit the result graph and calculate the purity of the target protein.

The results are shown in Table 2. Overall, as the length of the flexible linking peptide increased, the thermal stability of the antibody also became better, i.e., the difference with the protein in a state at 4 °C was smaller. The candidate molecule constructed with the flexible linking peptide of 60 amino acids in length exhibited the best thermal stability when treated at 50 °C.

TABLE 2
Comparison of thermal stability of candidate molecules
constructed by flexible linking peptides of different lengths

| | Difference | Difference |
|---|---|---|
| Number of | in purity | in purity |
| amino acids | between 37 °C | between 50 °C |
| of flexibly | and 4 °C heat | and 4 °C heat |
| linking peptides | treatment (%) | treatment (%) |
| 26 | 2.95 | 8.21 |
| 32 | 2.62 | 4.87 |
| 45 | 1.94 | 3.52 |
| 60 | 1.78 | 2.90 |
| 80 | 0.83 | 3.19 |

### 1.3 Effect of Different Lengths of Flexible Linking Peptides on Antigen Binding Ability

The binding ability of the purified antibodies (i.e., candidate molecules) in part 1.1 above to CD3 antigen was measured by ELISA. The brief procedure was as follows: 100 μL of CD3 antigen (Sino Biologics, SEK10981) at a concentration of 0.5 μg/mL was coated on an ELISA plate and placed at 4 °C overnight. After washing with PBS buffer containing 2% BSA, the plates were washed three times using PBS containing 2% BSA after blocked at room temperature for 1 hour. Then, 100 μL of antibody containing the gradient dilution was added and incubated for 1 hour at room temperature followed by 3 washes, and 100 μL of horseradish peroxidase-labeled sheep anti-human IgG secondary antibody (Bethyl Laboratories, Inc., 1:5000 dilution) was added and incubated for 1 hour at room temperature. After 3 washes, 100 μL of TMB chromogenic solution was added to develop the color, and the absorbance value at 450 nm was read on an enzyme marker.

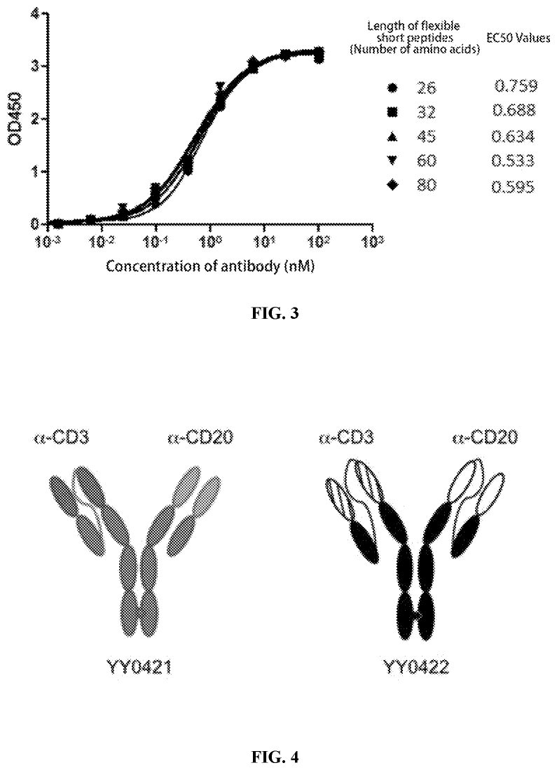

The results are shown in FIG. 3. Broadly speaking, as the length of the flexible linking peptide increased, the EC50 value decreased, i.e., the binding capacity increased. Among them, the candidate molecule constructed with the flexible linking peptide of 60 amino acids in length exhibited the best antigen binding ability (smallest EC50 value).

Comprehensively considering the effects of different lengths of flexible linking peptides on the expression level, thermal stability and antigen binding ability, we chose the flexible linking peptide of 60 amino acids in length for the subsequent construction of bispecific antibodies.

### Example 2. Preparation of Bispecific Antibodies

The DNA fragment encoding the CD3-binding moiety (anti-CD3 single chain Fab molecule, amino acid sequence SEQ ID NO: 6) and the DNA fragments encoding the anti-CD20 antibody light chain and heavy chain (amino acid sequences SEQ ID NO: 7 and SEQ ID NO: 8, respectively) were synthesized and cloned into eukaryotic expression vector pcDNA (purchased from Life Technologies, Inc.) using recombinant DNA such as overlapping PCR techniques, and then the obtained expression vector containing the DNA sequence encoding the CD3-binding moiety, the expression vector containing the DNA sequence encoding the light chain of anti-CD20 antibody and the expression vector containing the DNA sequence encoding the heavy chain of anti-CD20 antibody were mixed in a mass ratio of 1.5:2:1. The DNA-transfection reagent complex was prepared by mixing 200 μg of the above expression vector mixture with transfection reagent PEI (polyethylenimine) at a mass to volume ratio of 1:2.5, and added dropwise to 200 mL of 293F cells in logarithmic growth phase. The cell supernatant was collected 5-7 days after transfection, filtered through a 0.45 μm membrane, and then the supernatant was added to an affinity chromatography gel column (Protein A) washed with binding buffer (12.15 g of Tris (Tris(hydroxymethyl)aminomethane) dissolved in appropriate amount of sterilized water, pH adjusted to 7.0, 8.78 g of NaCl added, volume balanced to 1 L), and then eluted with eluent (7.5 g of glycine dissolved in appropriate amount of double-distilled water, pH adjusted to 3.5, 8.78 g of NaCl added, volume balanced to 1 L). The elution fraction was neutralized with 1M Tris-HCl (Tris(hydroxymethyl)aminomethane-HCl) pH 9.0 to obtain the bispecific antibody of the present invention YY0421. In a similar manner, the bispecific antibody of the present invention YY0422 was prepared, wherein the light and heavy chains of the CD20-binding moiety were linked by a linking peptide (the amino acid sequence of the CD3-binding moiety is shown in SEQ ID NO: 9, and the amino acid sequence of the CD20-binding moiety is shown in SEQ ID NO: 10). Schematic structures of the bispecific antibodies YY0421 and YY0422 are shown in FIG. 4.

### Example 3 Western Blotting Assay

### 3.1 Assay for Ability of the Bispecific Antibody of the Present Invention to Recognize CD3

Jurkat cells with healthy cell morphology and 80% fusion rate were collected, centrifuged and the supernatant discarded. Cell lysate was added, denatured at 95 °C for 10 minutes, and then cell proteins were separated by polyacrylamide gel electrophoresis. The proteins were transferred to nitrocellulose membranes (NC membranes) using the semi-dry transfer method and blocked with 5% skim milk for 1 hour at room temperature. Antibodies YY0421, YY0422 of the present invention or control antibody REGN1979 (a CD3 and CD20 bispecific antibody, see WO2014047231 and US20170355767) were added, incubated for 1 hour at room temperature, washed with PBST, and then a horseradish peroxidase labelled secondary antibody was added and incubated for 1 hour at room temperature. After PBST washing, the color was developed using chemiluminescent reagent and developed in the dark room.

The results of the Western blotting assay are shown in FIG. 5a. The results indicate that the bispecific antibodies YY0421 and YY0422, which target both human CD3 and CD20, can effectively recognize the CD3 antigen at the protein level.

### 3.2 Assay for Ability of the Bispecific Antibody of the Present Invention to Recognize CD20

Raji cells with healthy cell morphology and 80% fusion rate were collected, centrifuged and the supernatant was discarded. Cell lysate was added, denatured at 95 °C for 10 minutes, and then cell proteins were separated by polyacrylamide gel electrophoresis. The proteins were transferred to nitrocellulose membranes (NC membranes) using the semi-dry transfer method and blocked with 5% skim milk for 1 hour at room temperature. Antibodies YY0421, YY0422 of the present invention or control antibody REGN1979 were added, incubated for 1 hour at room temperature, washed with PBST, and then a horseradish peroxidase labelled secondary antibody was added and incubated for 1 hour at room temperature. After PBST washing, the color was developed using chemiluminescent reagent and developed in the dark room.

The results of the Western blotting assay are shown in FIG. 5b. The results indicate that the bispecific antibodies YY0421 and YY0422, which target both human CD3 and CD20, can effectively recognize the CD20 antigen at the protein level.

### Example 4. Flow Cytometry for Detecting the Binding of Bispecific Antibodies of the Present Invention to CD3 and CD20 on Cell Surface

Human Raji and Jurkat cells were inoculated in 6-well plates, incubated for 20 hours and then digested by trypsin (without EDTA) and collected, centrifuged at 1000 rpm for 5 minutes at room temperature and washed twice with PBS and incubated with YY0421, YY0422 or control antibody REGN1979 for 1 hour on ice, washed with PBS and incubated with FITC-labeled rabbit anti-human IgG (Fab) for 1 hour on ice and detected by flow cytometry.

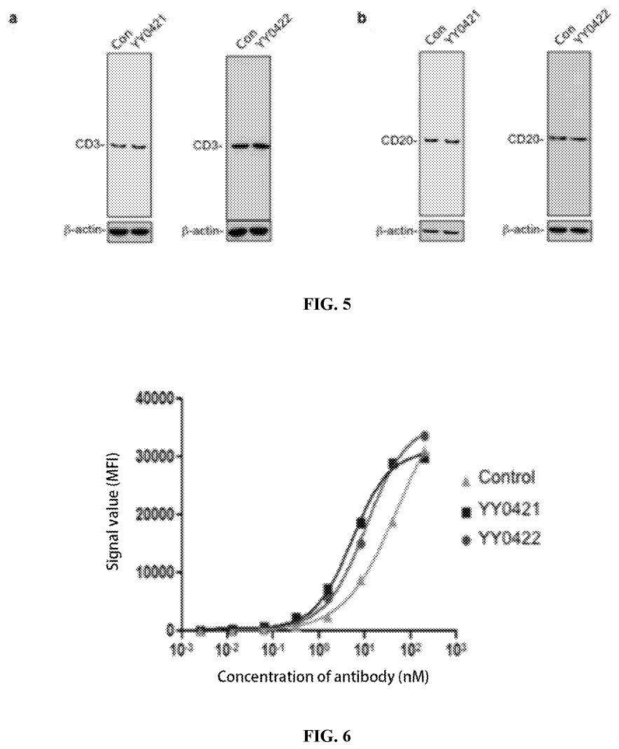

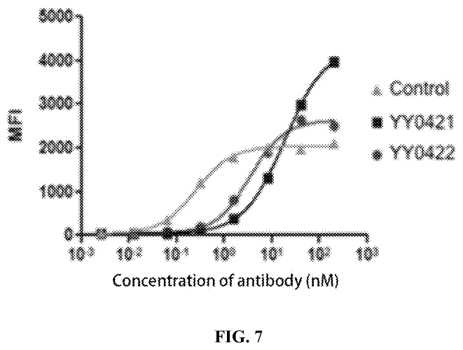

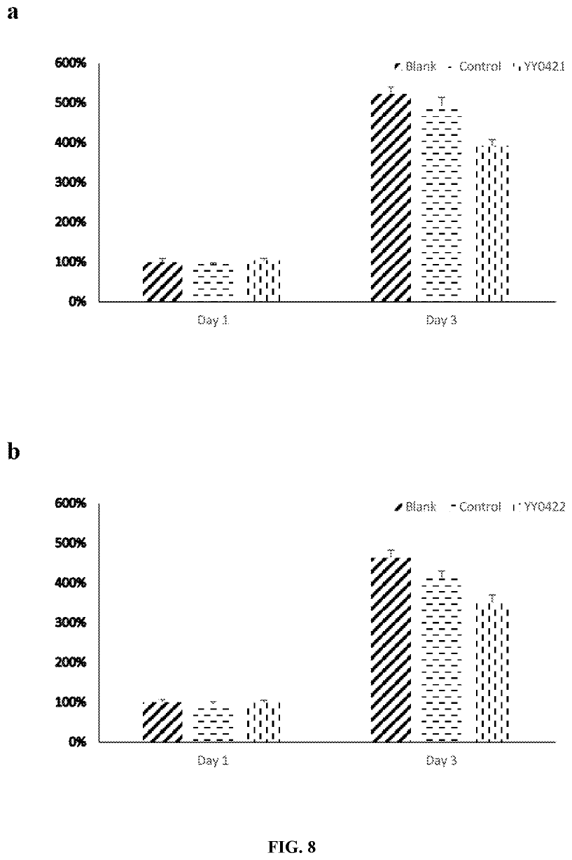

The results of the flow cytometry assay are shown in FIGS. 6 and 7. The results indicate that the bispecific antibodies YY0421 and YY0422 of the present invention, which target both human CD3 and CD20, had a high affinity for human CD3 (FIG. 6) and CD20 (FIG. 7) on the cell surface.

### Example 5. Bispecific Antibodies of the Present Invention Mediate Killing of Tumor Cells by CD8⁺ T Cells

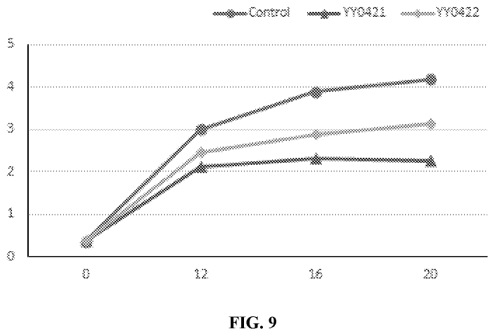

Morphologically healthy human Daudi cells in an amount of 5×10³, which were stained with CFSE of a final concentration of 5 M, were inoculated in a 96-well plate. After overnight starvation with medium containing 0.5% fetal bovine serum, CD8⁺ T cells were added at an E/T ratio of 5:1. The experiments were performed in two batches, each batch dividing the cells into three groups (each group comprised three wells). In the first batch, culture medium (Blank in FIG. 8a), control antibody REGN1979 (Control in FIG. 8a), and antibody YY0421 of the invention were added separately. In the second batch, culture medium (Blank in FIG. 8b), control antibody REGN1979 (Control in FIG. 8b), and antibody YY0422 of the present invention were added separately. Then, the incubation was continued in the cell incubator for 4 hours. The supernatant and cell suspension were collected, centrifuged, with supernatant discarded and then resuspended with PBS, followed by the addition of 1 μg/mL PI, and the ratio of CFSE and PI double-positive cells to CFSE-positive cells was detected by flow cytometry to detect the ability of bispecific antibodies to mediate CD8+T killing of tumor cells.

The killing results are shown in FIG. 8. From the results, it was clear that the bispecific antibodies YY0421 and YY0422 of the present invention, which target both human CD3 and CD20, can effectively mediate the killing of Daudi cells by T cells.

### Example 6. Assay for Detecting Transplanted Tumors in Nude Mice

5×10⁷ human Daudi cells in logarithmic growth stage with good morphology were injected subcutaneously into the anterior axilla of SPF grade BALB/C nude mice, and 10 μg each of PBMC cells (2.5×10⁸, purchased from ATCC (American Type Culture Collection)) and a bispecific antibody of the present invention or control antibody REGN1979, were injected on day 6 after injection. The maximum longitudinal diameter and maximum transverse diameter of the subcutaneous tumors were measured daily, the volume of the tumors was calculated, and the diet and body weight of the nude mice were observed and recorded. On day 28, all mice were executed by cervical dissection, and the tumors were then separated, photographed and weighed.

The results of the assay for detecting transplanted tumors in nude mice are shown in FIG. 9. The results indicated that the bispecific antibody of the present invention targeting both human CD3 and CD20 had good biological activity and can significantly inhibit the proliferation of human Daudi cell-induced subcutaneous tumors in nude mice.

Some of the amino acid sequences mentioned herein are as follows.

### YY0421 Amino Acid Sequence of the CD3-Binding Moiety SEQ ID NO: 6

DIQLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFG
AGTKLELKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGECGGGGSGGGGSGGGGSGGGGSGGGGSGGGGSGG
GGSGGGGSGGGGSGGGGSGGGGSGGGGSDIKLQQSGAELARPGASVKMS
CKTSGYTFTRYTMHWVKQRPGQGLEWIGYINPSRGYTNYNQKFKDKATL
TTDKSSSTAYMQLSSLTSEDSAVYYCARYYDDHYCLDYWGQGTTLTVSS
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLG

### YY0421 Amino Acid Sequence of Light Chain of the CD20-Binding Moiety SEQ ID NO: 7

EIVLTQSPATLSLSPGERATLSCRASQSVSSYLAWYQQKPGQAPRLLIY
DASNRATGIPARFSGSGSGTDFTLTISSLEPEDFAVYYCQQRSNWPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

### YY0421 Amino Acid Sequence of Heavy Chain of the CD20-Binding Moiety SEQ ID NO: 8

EVQLVESGGGLVQPGRSLRLSCAASGFTFNDYAMHWVRQAPGKGLEWVS
TISWNSGSIGYADSVKGRFTISRDNAKKSLYLQMNSLRAEDTALYYCAK
DIQYGNYYYGMDVWGQGTTVTVSSASTKGPSVFPLAPCSRSTSESTAAL
GCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLF
PPKPKDTLMISRTPEVTCVVVDVSQEDPEVQFNVVYVDGVEVHNAKTKP
REEQFNSTYRVVSVLTVLHQDWLNGKEYKCKVSNKGLPSSIEKTISKAK
GQPREPQVCTLPPSQEEMTKNQVSLSCAVKGFYPSDIAVEWESNGQPEN
NYKTTPPVLDSDGSFFLVSRLTVDKSRWQEGNVFSCSVMHEALHNHYTQ
KSLSLSLG

### YY0422 Amino Acid Sequence of the CD3-Binding Moiety SEQ ID NO: 9

DIQLTQSPAIMSASPGEKVTMTCRASSSVSYMNWYQQKSGTSPKRWIYD
TSKVASGVPYRFSGSGSGTSYSLTISSMEAEDAATYYCQQWSSNPLTFG
AGTKLELKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGECGGGGSGGGGSGGGGSGGGGSGGGGSGGGGSGG
GGSGGGGSGGGGSGGGGSGGGGSGGGGSDIKLQQSGAELARPGASVKMS
CKTSGYTFTRYTMHWVKQRPGQGLEWIGYINPSRGYTNYNQKFKDKATL
TTDKSSSTAYMQLSSLTSEDSAVYYCARYYDDHYCLDYWGQGTTLTVSS
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLG

### YY0422 Amino Acid Sequence of the CD20-Binding Moiety SEQ ID NO: 10

EIVLTQSPATLSLSPGERATLSCRASQSVSSYLAWYQQKPGQAPRLLIY
DASNRATGIPARFSGSGSGTDFTLTISSLEPEDFAVYYCQQRSNWPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGECGGGGSGGGGSGGGGSGGGGSGGGGSGGGGSG
GGGSGGGGSGGGGSGGGGSGGGGSGGGGSEVQLVESGGGLVQPGRSLRL
SCAASGFTFNDYAMHWVRQAPGKGLEWVSTISWNSGSIGYADSVKGRFT
ISRDNAKKSLYLQMNSLRAEDTALYYCAKDIQYGNYYYGMDVWGQGTTV
TVSSASTKGPSVFPLAPCSRSTSESTAALGCLVKDYFPEPVTVSWNSGA
LTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVV
VDVSQEDPEVQFNWYVDGVEVHNAKTKPREEQFNSTYRVVSVLTVLHQD
WLNGKEYKCKVSNKGLPSSIEKTISKAKGQPREPQVCTLPPSQEEMTKN
QVSLSCAVKGFYPSDIAVEWESNGQPENNYKTTPPVLDSDGSFFLVSRL
TVDKSRWQEGNVFSCSVMHEALHNHYTQKSLSLSLG

### Tandemly Repeated Unit Sequence SEQ ID NO: 11

GGGGS

1. A bispecific antibody comprising a first moiety for binding to CD3 and a second moiety for binding to CD20, wherein the first moiety is an anti-CD3 single chain Fab molecule comprising a Fab light chain and a Fab heavy chain with C-terminal of the Fab light chain being connected to the N-terminal of the Fab heavy chain by a linking peptide having a length of 60 amino acids; the second moiety comprises a light chain and a heavy chain of an anti-CD20 antibody.

2. The bispecific antibody according to claim 1, wherein the linking peptide of the first moiety comprises a tandemly repeated GGGGS sequence as set forth in SEQ ID NO: 11.

3. The bispecific antibody according to claim 2, wherein the linking peptide of the first moiety consists of 12 tandem repeats of GGGGS and has the amino acid sequence as set forth in SEQ ID NO: 4.

4. The bispecific antibody according to claim 1, wherein the first moiety for binding to CD3 comprises an amino acid sequence as set forth in SEQ ID NO: 6 or 9.

5. The bispecific antibody according to claim 4, wherein the first moiety for binding to CD3 comprises the amino acid sequence as set forth in SEQ ID NO: 6.

6. The bispecific antibody according to claim 4, wherein the first moiety for binding to CD3 comprises the amino acid sequence as set forth in SEQ ID NO: 9.

7. The bispecific antibody according to claim 1, wherein the light chain of the anti-CD20 antibody comprises an amino acid sequence as set forth in SEQ ID NO: 7, and the heavy chain of the anti-CD20 antibody comprises an amino acid sequence as set forth in SEQ ID NO: 8.

8. The bispecific antibody according to claim 7, wherein the first moiety for binding to CD3 comprises the amino acid sequence as set forth in SEQ ID NO: 6.

9. The bispecific antibody according to claim 1, wherein the second moiety is an anti-CD20 single chain Fab molecule comprising a Fab light chain and a Fab heavy chain, wherein C-terminal of the Fab light chain is connected to N-terminal of the Fab heavy chain by a linking peptide having a length of 60 amino acids.

10. The bispecific antibody according to claim 9, wherein the linking peptide of the second moiety comprises a tandemly repeated GGGGS sequence as set forth in SEQ ID NO: 11.

11. The bispecific antibody according to claim 10, wherein the linking peptide of the second moiety consists of 12 tandem repeats of GGGGS and has the amino acid sequence as set forth in SEQ ID NO: 4.

12. The bispecific antibody according to claim 9, wherein the second moiety for binding to CD20 comprises an amino acid sequence as set forth in SEQ ID NO: 10.

13. The bispecific antibody according to claim 12, wherein the first moiety for binding to CD3 comprises the amino acid sequence as set forth in SEQ ID NO: 9.

14. The bispecific antibody according to claim 1, wherein the first moiety and the second moiety are covalently bound.

15. The bispecific antibody according to claim 1, wherein the bispecific antibody comprises Fc fragments having a knobs-into-holes structure.

16. A pharmaceutical composition comprising the bispecific antibody of claim 1 and a pharmaceutically acceptable carrier.

17. The pharmaceutical composition according to claim 16, wherein the first moiety for binding to CD3 comprises an amino acid sequence as set forth in SEQ ID NO: 6, the light chain of the anti-CD20 antibody comprises an amino acid sequence as set forth in SEQ ID NO: 7, and the heavy chain of the anti-CD20 antibody comprises an amino acid sequence as set forth in SEQ ID NO: 8.

18. The pharmaceutical composition according to claim 16, wherein the first moiety for binding to CD3 comprises an amino acid sequence as set forth in SEQ ID NO: 9, and the second moiety for binding to CD20 comprises an amino acid sequence as set forth in SEQ ID NO: 10.

19. A method of treating a B-cell related disease in a patient comprising administering to the patient a therapeutically effective amount of the bispecific antibody of claim 1.

20. The method according to claim 19, wherein the B-cell related disease is B-cell leukemia or B-cell lymphoma.


SEQUENCE LISTING (11 sequences)

SEQ ID NO: 1 - protein, 26 aa, synthetic construct
  REGION 1..26: note = Linking peptide
GGGSGGSGGSGGSGGSGGSGGSGGSG

SEQ ID NO: 2 - protein, 32 aa, synthetic construct
  REGION 1..32: note = Linking peptide
GGGGSGGGGSGGGGSGGGGSGGGGSGGGGSGG

SEQ ID NO: 3 - protein, 45 aa, synthetic construct
  REGION 1..45: note = Linking peptide
GGGGSGGGGSGGGGSGGGGSGGGGSGGGGSGGGGSGGGGSGGGGS

SEQ ID NO: 4 - protein, 60 aa, synthetic construct
  REGION 1..60: note = Linking peptide
GGGGSGGGGSGGGGSGGGGSGGGGSGGGGSGGGGSGGGGSGGGGSGGGGSGGGGSGGGGS

SEQ ID NO: 5 - protein, 80 aa, synthetic construct
  REGION 1..80: note = Linking peptide
GGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 6 - protein, 718 aa, synthetic construct
  REGION 1..718: note = CD3-binding moiety
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 214 aa, synthetic construct
  REGION 1..214: note = CD20-binding moiety light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 448 aa, synthetic construct
  REGION 1..448: note = CD20-binding moiety heavy chain
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 9 - protein, 718 aa, synthetic construct; identical to SEQ ID NO: 6
  REGION 1..718: note = CD3-binding moiety

SEQ ID NO: 10 - protein, 722 aa, synthetic construct
  REGION 1..722: note = CD20-binding moiety
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSGGGGSGGGGSGGGGSGGGGSGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 11 - protein, 5 aa, synthetic construct
  source 1..5: note = Tandemly repeated unit sequence
GGGGS